(19) United States
(12) Patent Application Publication
(10) Pub. No.: US 2012/0156491 A1
(43) Pub. Date: Jun. 21, 2012

(54) SERS Reporter Molecules and Methods

(75) Inventors: Felicia Tam, Sunnyvale, CA (US); Marcelo E. Piotti, Freemont, CA (US); Richard G. Freeman, Mountain View, CA (US)

(73) Assignee: CABOT SECURITY MATERIALS INC., Mountain View, CA (US)

(21) Appl. No.: 13/163,392

(22) Filed: Jun. 17, 2011

Related U.S. Application Data
(60) Provisional application No. 61/356,365, filed on Jun. 18, 2010.

(51) Int. Cl. B32B 5/16; C07D 401/14; C07D 213/16; C07D 471/04; C07C 211/61

(52) U.S. Cl. 428/402; 544/350; 564/427; 546/285; 546/255; 534/653

(57) ABSTRACT

A SERS tag comprising a core comprising at least two aggregated particles of a SERS enhancing material wherein the contact point between the particles defines a crevice; and a reporter molecule having a length sufficiently short to fit into the crevice and a conjugated path length which is as large as possible, provided the overall reporter molecule length is maintained sufficiently short to fit into the crevice.

### BACKGROUND

Certain spectroscopy techniques feature the enhancement of a spectroscopic signal through electromagnetic interaction at a surface. Representative surface enhanced spectroscopic (SES) techniques include, but are not limited to surface enhanced Raman spectroscopy (SERS) and surface enhanced resonance Raman spectroscopy (SERRS). In SERS or SERRS, a metal or other enhancing surface will couple electromagnetically to incident electromagnetic radiation and create a locally amplified electromagnetic field that leads to 10²- to 10⁹-fold or greater increases in the Raman scattering of a SERS active molecule situated on or near the enhancing surface. The output in a SERS experiment is the fingerprint-like Raman spectrum of the SERS active molecule. A SERS active molecule may be alternatively referred to herein as a reporter molecule or reporter.

SERS and other SES techniques can be implemented with particles such as nanoparticles. For example, gold is a SERS enhancing surface, and gold colloid may be suspended in a mixture to provide for enhanced Raman spectrum detection. SERS may also be performed with more complex SERS-active nanoparticles, for example SERS nanotags, as described in U.S. Pat. No. 6,514,767, U.S. Pat. No. 6,861,263, U.S. Pat. No. 7,443,489 and elsewhere.

Although SERS techniques leverage the enhancement phenomenon described above, it is still important for many diverse implementations that the Raman spectrum or signal be as great or bright as possible. Certain known reporter molecules do not return as strong a signal as may be desired in solution, when used with a colloidal enhancing surface, when incorporated into a more complex tag type particle, on an enhancing substrate or otherwise.

The present invention is directed toward overcoming one or more of the problems discussed above.

### SUMMARY

One embodiment is a SERS reporter molecule comprising a reporter molecule having a length sufficiently short to fit into a crevice formed by the enhancing surfaces of adjacent enhancement particles; and a conjugated path length which is as large as possible, provided the overall molecule length is maintained sufficiently short to fit into the crevice formed by the enhancing surfaces of adjacent enhancement particles.

Another embodiment is a SERS tag comprising a core comprising at least two aggregated particles of a SERS enhancing material wherein the contact point between the particles defines a crevice; and a reporter molecule having a length sufficiently short to fit into the crevice and a conjugated path length which is as large as possible, provided the overall reporter molecule length is maintained sufficiently short to fit into the crevice.

Another embodiment is a method of selecting a SERS reporter molecule to optimize the enhancement of the SERS signal provided by said reporter molecule upon excitation in association with a particulate or colloidal enhancing surface, said method comprising: selecting or fabricating a reporter molecule length to be sufficiently short to fit into a crevice formed by the enhancing surfaces of adjacent enhancement particles; and selecting or fabricating the reporter molecule to have as high a conjugated path length as is possible provided the overall molecule length is maintained sufficiently short to fit into the crevice formed by the enhancing surfaces of adjacent enhancement particles.

### BRIEF DESCRIPTION OF THE DRAWINGS

FIG. 1 illustrates SERS enhancement vs. reporter molecule length. (top) Structures of the reporter molecules studied, along with their names and lengths. (bottom) SERS enhancements using 785-nm excitation of various reporter molecules on 90-nm Au colloid that was aggregated in the presence of the reporter.

FIG. 2 illustrates a simulated 90 nm Au colloid dimer hot spot. (left) Electric near-field enhancement created in the junction of a touching 90 nm Au colloid dimer. (right) Electric near-field enhancement as a function of the distance, d, from the junction.

FIG. 3 illustrates curve fitting of SERS spectra. Comparison of an experimental SERS spectrum of D1 on 90-nm Au colloid and the curve-fit to this spectrum generated by the algorithm used in analysis.

FIG. 4 illustrates enhancements of D-set of reporter molecules. (top) Schematic of the reporter molecules studied, along with their names and lengths. SERS enhancement factors of the (middle) 1600 cm⁻¹ and (bottom) 1000 cm⁻¹ peak using 633, 785, and 1064-nm excitation of the various reporter molecules on 90-nm Au colloid that was aggregated in the presence of the reporter. Lines are power law fits to the data points.

FIG. 5 illustrates absorbance spectra from each series of measurements on (left) D1 and (right) D6. (top) Spectra taken before (red), during (green), and after (blue) SERS measurements show little variability over the course of the measurements. (bottom) Sample-to sample variation demonstrates differences in the aggregation state of each sample. The data are taken from two different instruments, leaving a small gap where spectral data is unavailable. A few of the NIR spectra are missing due to some temporary instrument problems.

FIG. 6 illustrates enhancements of P-set of reporter molecules. (top) Schematic of the reporter molecules studied, along with their names and lengths. (bottom) SERS enhancement factors of the 1600 cm⁻¹ peak using 633, 785, and 1064-nm excitation of the various reporter molecules on 90-nm Au colloid that was aggregated in the presence of the reporter. Lines are power law fits to the data points.

FIG. 7 the distance of the molecule from the junction as a function of molecule length for different sizes of colloid. This plot assumes that the Au aggregates are perfect spheres with one point of contact, and that the molecules align themselves perpendicular to the plane separating the two spheres.

FIG. 8 illustrates maximum SERS enhancement on different sized colloid. (Left) SERS enhancement as a function of molecule length using 633, 785, and 1064-nm excitation on 40, 60, 90, and 120-nm Au colloid. Lines are power-law fits to help guide the eye. (Right) The same data plotted for each colloid size and on a log scale with linear fits to guide the eye.

FIG. 9 illustrates calculated extinction cross-sections of touching Au colloid dimers immersed in water. (Top) Extinction of a dimer of 90-nm Au nanoparticles with an overlap, D, of 1 nm to simulate the contact area of the two nanoparticles. Two different dimer orientations relative to the polarization of the incoming light are considered. (Bottom) Calculated extinction of dimer particles with different overlaps; a spectrum of the extinction expected from two non-interacting nanospheres is included for comparison.

FIG. 10 illustrates SERS enhancements of 1600 cm⁻¹ Raman peak. (Left) Enhancement at the center of a molecule oriented parallel to the dimer axis and bound to each nanoparticle surface at either end for dimers of 40, 60, 90, and 120-nm Au nanospheres and using 633, 785, and 1064-nm excitation wavelengths. (Right) Electromagnetic near-field enhancement of a (top) 40 nm dimer with D=1.33 nm, and a (bottom) 120 nm dimer with D=4 nm.

FIG. 11 illustrates the structure and size of reporters used in one aspect of the study.

FIG. 12 illustrates the synthesis of a selected reporter molecule.

FIG. 13 illustrates the Intensity of highest Raman peak for each reporter; 0.1 M solutions in DMF. Values corrected for background.

FIG. 14 illustrates a comparison of SERS intensity for the highest peak for each reporter.

FIG. 15 is a SEM images of Klarite substrates.

FIG. 16 illustrates a comparison of colloid and substrate SERS intensity for the highest peak for each reporter.

FIG. 17 is a schematic representation of the positioning of different sized reporters around the contact area of aggregated particles.

FIG. 18 illustrates enhancement factors for selected reporter molecules.

### DETAILED DESCRIPTION

Unless otherwise indicated, all numbers expressing quantities of ingredients, dimensions reaction conditions and so forth used in the specification and claims are to be understood as being modified in all instances by the term “about”.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

Known prior art marking or detection methods which utilize SERS typically rely upon a reporter molecule or dye with known SERS-active characteristics. For example, a known SERS-active chemical can be added as a dye to mark fuel and a subsequent SERS spectrum obtained when the SERS-active dye is associated with a SERS-active metal particle or substrate. Only a limited number of SERS active chemicals are known.

Many of the examples herein are described with respect to SERS. It must be noted however that the methods, compositions and reporters disclosed herein are equally applicable to SERRS, SEHRS, SEF, SEHRRS, SHG, SEIRA, SPASERS, or other surface enhanced or plasmon enhanced SES techniques.

One aspect of SERS that is often overlooked when carrying out fundamental SERS studies is the role of the Raman-active reporter molecule. There are several requirements placed upon the molecule if it is going to generate a large or bright SERS signal.
The molecule should have an inherently large Raman cross-section.
The molecule must reside on or very close to the enhancing surface.
Maximum enhancement is obtained for vibrational modes that are oriented parallel to the polarization of the exciting radiation.
Maximum enhancements are obtained for molecules which reside in “hot spots” on the SERS surfaces.

This last detail results in certain types of molecules performing better or worse as practical reporters than would be expected based upon Raman cross-section alone.

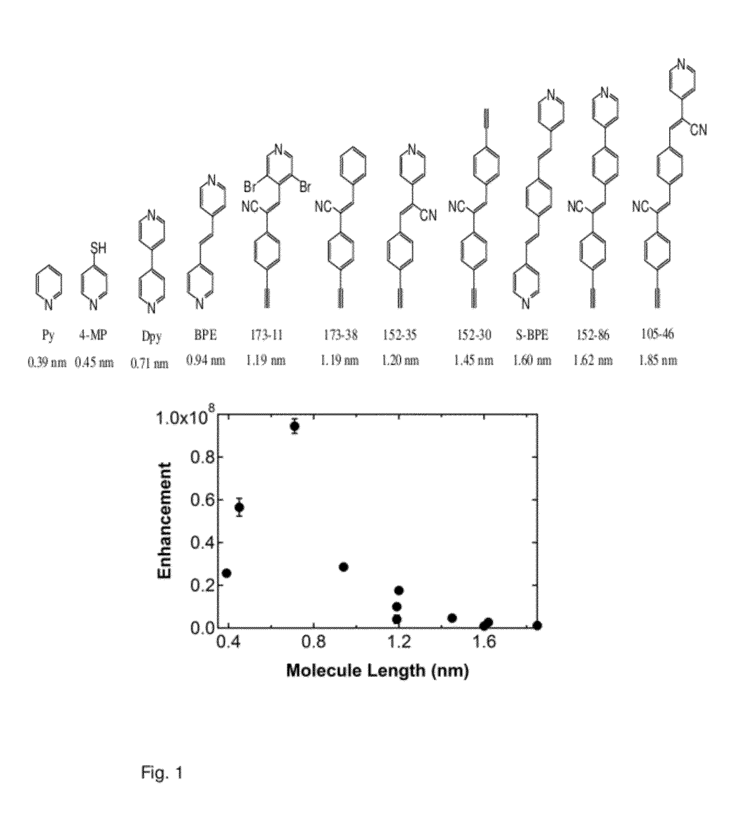

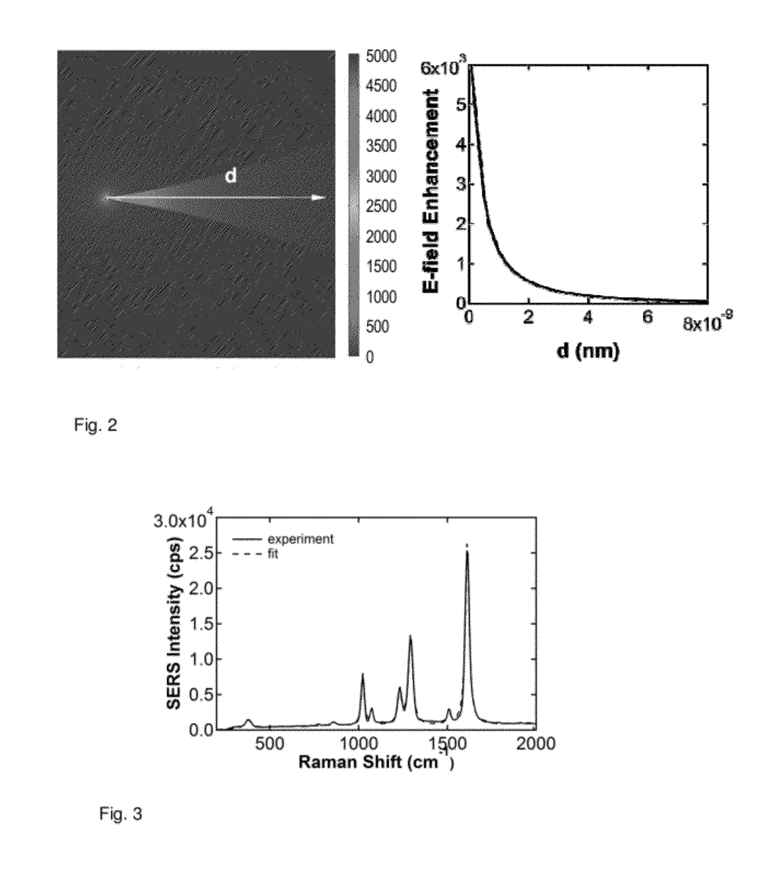

It has been determined that there is a correlation between the size of a reporter molecule and the subsequent SERS enhancement achievable on Au nanospheres aggregated in situ. For example, FIG. 1 shows that as the length of the reporter molecule increases, the calculated enhancement factor decreases. In contrast, when SERS signals from these same molecules are measured on Klarite™ substrates, enhancement was found to be independent of reporter molecule length. The relationship between SERS enhancement and molecule size on aggregated Au colloid has been explained by the ability of the molecule to fit into the regions of enhanced electromagnetic near fields, or “hotspots,” created by the aggregated Au nanospheres; Mcmahon, J. M.; Henry, A. I.; Wustholz, K. L.; Natan, M. J.; Freeman, R. G.; Van Duyne, R. P.; Schatz, G. C. Gold nanoparticle dimer plasmonics: finite element method calculations of the electromagnetic enhancement to surface-enhanced Raman spectroscopy. Analytical and Bioanalytical Chemistry 2009, 394 (7), 1819-1825. The hotspots created by aggregating Au colloid are located in the crevices formed by the aggregated particles, and the enhanced near fields therein rapidly decrease as the distance from the crevice increases (See for example the Simulation of FIG. 2).

Although reporter molecules that are small in size are able to access the hotspots, larger molecules have more difficulty fitting into a crevices. Therefore, the near field intensity experienced by larger molecules is relatively lower, resulting in decreased SERS enhancement factors. In contrast, Klarite Au surfaces give lower overall enhancement, due to a broader variety of surface geometries. The “hotspots” on these surfaces are not as hot, or as geometrically restrictive. These results demonstrate that SERS reporter molecule size is a crucial factor to be considered when optimizing SERS surfaces, particularly as the enhancement factors become larger.

Although the overall trend of decreasing enhancement with increasing molecule size is clear, upon closer inspection, there are some inconsistencies. For instance, the smallest molecules studied did not have the largest enhancements. In addition, there were a number of molecules studied with approximately the same length that had significantly different enhancements. However, when only molecules with the same terminal functional groups and similar structures are considered, the trend appears to be more consistent.

The chemical structure of the reporter molecule could influence the resultant SERS enhancement in a number of ways. First, the SERS enhancements in the experiments described below arise from the hotspots formed by Au nanospheres that are aggregated in the presence of the reporter either by the molecule itself or by salt that is added to the solutions. Different chemical structures of the molecules may induce different degrees or kinds of aggregation. For instance some molecules may favor forming small aggregates of only a few particles, while others may favor forming large aggregates of tens of particles. The kinds of aggregates formed are probably determined by the mechanism by which the molecule induces aggregation (e.g. electrostatic interactions, covalent bonding to the colloid surface, etc.). Different functional groups within the molecule may also cause the molecule to orient itself differently in the crevices. The molecular orientation in relation to the gradient of the enhanced electromagnetic near field determines which of the molecule's Raman modes are enhanced, and to what degree. The functional groups will also affect the affinity of the molecule to the colloid surface, thereby affecting the number of molecules contributing to the SERS signal.

The experiments discussed below were carried out in order to isolate the impact of molecule size on the resultant SERS enhancements for selected SERS system. Two different sets of molecules were carefully chosen so that the molecules within each set contained the same terminal functional groups and no other functional groups which would compete for competition to Au surfaces. The molecules were also similar in structure so that similar Raman modes in each molecule could be compared. SERS measurements were taken using three different excitation wavelengths to rule out the possibility that molecular resonances were affecting the results. To probe the importance of the hotspot accessibility more, the experiments were also performed on Au nanospheres of different sizes. The different radii of curvature in these samples should allow reporter molecules of different sizes to penetrate into the hotspots in varying degrees. The spectra were then analyzed by fitting each SERS spectrum as a sum of Lorentzian functions and a third-order polynomial background. This analysis is an improvement over cruder background subtraction method used for the earlier experiments that was not as accurate for spectra with low signal to noise or high background fluorescence. This study supports the conclusion that the SERS enhancement decreases with increasing molecule size when measured on Au nanospheres aggregated in situ.

### EXAMPLES

The following examples are provided for illustrative purposes only and are not intended to limit the scope of the invention.

### Example 1

Procedure

### Reporter Molecules

The reporter molecules used in the described examples are listed in Table 1, along with abbreviations which will be used throughout the rest of this disclosure. Molecules D1, D2, D4, and P1 were purchased from Sigma Aldrich. Molecule D6 was purchased from Exciton. Other molecules were synthesized by applicant.

TABLE 1
Reporter molecules and abbreviations

| Abbreviation | Molecule |
|---|---|
| D1 | 4,4′-dypyridyl |
| D2 | trans-1,2-bis(4-pyridyl)-ethylene |
| D3 | 1,2-di(4-pyridyl) acetylene |
| D4 | 4-pyridinealdazine |
| D5 | 1-(4-[4-pyridyl]phenyl)-2-(4-pyridyl)ethylene |
| D6 | 1,4-Bis-(4-pyridyl-2-ethenyl) Benzene |
| P1 | 4-phenylpyridine |
| P2 | 2-phenyl-1-(4-pyridyl)ethylene |
| P3 | 1-(4-biphenyl)-2-(4-pyridyl)ethylene |
| P4 | (4-biphenyl)(4-pyridyl)acetylene |

### Normal Raman Measurements

Normal Raman measurements of the reporter molecules were made using solutions of the molecule dissolved in dimethylformamide (DMF). For the majority of the molecules the concentration was 0.1 M. For D1 the spectrum was measured at 1 M due to its low Raman cross-section. Larger molecules were measured at lower concentrations due to low solubility: P3 was measured at 0.025 M, while D6 and P4 were measured at 0.02 M.

### SERS and UV-vis-NIR Measurements

All the reporter molecules used were diluted to 0.1 mM in ethanol for the SERS measurements. SERS spectra of the reporters were acquired on aggregated Au colloid and UV-vis-NIR measurements were taken between SERS measurements to monitor possible changes to the colloid aggregation between SERS measurements at different wavelengths.

The first set of experiments monitored the SERS from the two different families of molecules. The Au colloid had a diameter of 94.4±5.5 nm (as measured by TEM) and an as-made concentration of 1.17×10¹⁰ particles/mL (0.1 g/L of Au). First, the optimal ratio of colloid to reporter volume was determined by introducing varying aliquots of the reporter solution to 1 mL of the colloid while monitoring the SERS intensity using 785-nm excitation and a 1 s integration time. If the reporter itself did not aggregate the colloid, 14 μL of 1 M NaCl was also introduced to the solution immediately after the reporter addition to induce aggregation. The SERS spectrum was monitored over time, and the maximum intensity observed was recorded. The optimal reporter volume was found to be 7 μL for each of the reporter molecules studied. This reporter to colloid ratio was used throughout the experiments.

Measurements were made by adding 10 μL of the reporter solution to 1.5 mL of as-made colloid, and vortexing the solution rapidly. If the reporter itself did not induce aggregation in the colloid, 20 μL of 1 M NaCl was added to the colloid solution immediately after the reporter addition. The SERS signal using 785-nm excitation was monitored while the sample was periodically vortexed. Once the SERS intensity reached a plateau, the solution was diluted by a factor of two. The following measurements were then made in order: UV-vis, NIR, SERS using 785-nm excitation, UV-vis, NIR, SERS using 1064-nm excitation, SERS using 633-nm excitation, UV-vis, and finally NIR. The samples were agitated with a pipette between each measurement. UV-vis and NIR measurements were made in 1 cm path length polystyrene cuvettes. The SERS measurements were made with a neat pyridine sample as a reference. A 750 μL aliquot of the colloid/reporter solution was transferred to a glass vial for SERS measurements, and then returned to the original sample and mixed before the next UV-vis and NIR measurement.

This series of absorbance and SERS measurements was completed between two and eight times for each reporter molecule, by at least two different people to ensure reproducibility. The exceptions are D5 and P4, which were somewhat unstable in solution, so that only two series of measurements made by one person were possible.

The second set of experiments involved looking for the maximum SERS enhancement factors using different excitation wavelengths and different colloid sizes. The Au colloid studied had diameters of 42.4±2.9, 59.1±3.8, 94.4±5.5, and 119.1±7.0 nm. For brevity, these colloid sizes will hereafter be referred to as 40, 60, 90, and 120 nm. Each of these colloids has an as-made Au concentration of 0.1 g/L. However, since the nanospheres are of different sizes, the particle concentrations will vary. The series of SERS and absorbance measurements were conducted as before, but this time multiple reporter volumes were used for each colloid, and the experiment was only carried out once for each reporter volume and colloid size.

### Spectral Analysis

Both the normal Raman and SERS spectra were analyzed using original procedures written using Igor Pro 6.1 (Wavemetrics, Inc.). The spectra were fit to the sum of a number of Lorentzian functions with various peak heights and a third-order polynomial background (See for example the representative curve fit of FIG. 3). The Lorentzian functions were used to fit the observed Raman peaks, and the polynomial background accounted for any background fluorescence that may have been present. The peak locations, amplitudes, and widths were derived from this analysis.

### Enhancement Calculation

To compare the SERS enhancements of different molecules, the analytical enhancement factor as presented by Le Ru, et al. was employed; Le Ru, E. C.; Blackie, E.; Meyer, M.; Etchegoin, P. G. Surface enhanced Raman scattering enhancement factors: a comprehensive study. Journal of Physical Chemistry C 2007, 111 (37), 13794-13803. Specifically, the SERS enhancement factor, EF, of a particular Raman peak is calculated by comparing the peak intensity of the normal Raman signal, I_{Raman}, to that of the SERS signal, I_{SERS}, using the equation

$$EF = \frac{{I}_{SERS}}{{I}_{Raman}} \times \frac{{C}_{Raman}}{{C}_{SERS}}$$

in which C_{Raman} and C_{SERS} are the reporter molecule concentrations used in the normal Raman and SERS measurements, respectfully. This calculation assumes that all of the reporter molecules used in the preparation of SERS samples are contributing to the SERS signal. Since the Raman spectrum of each molecule is unique, this study focuses on two peaks that are common to all of the molecules used, those near 1600 cm⁻¹ and 1000 cm⁻¹.

### Example 1

### Results and Discussion

Enhancement vs. Molecule Size

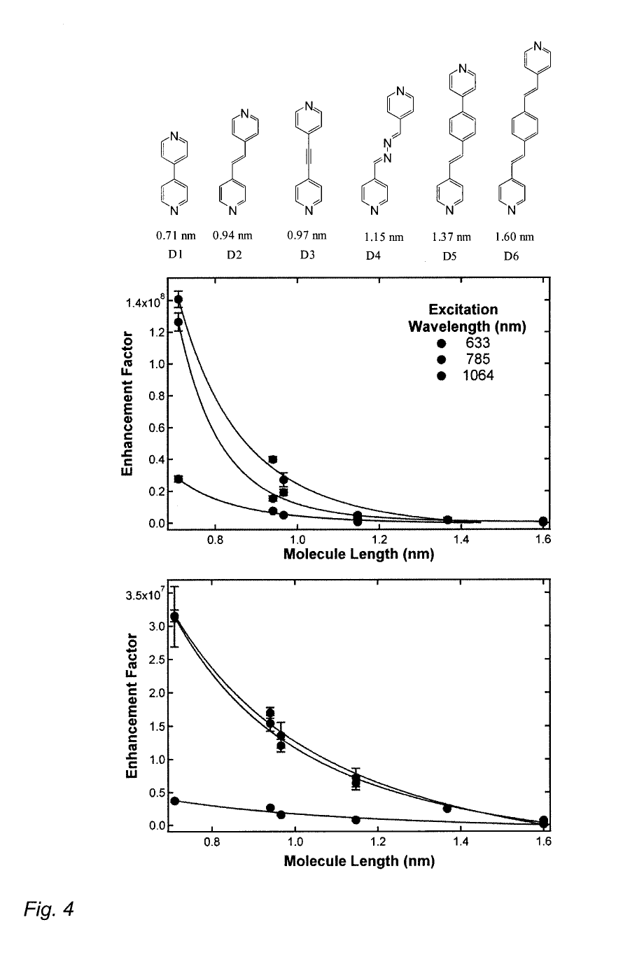

In order to isolate the impact of molecule size on the achievable SERS enhancements, two different sets of molecules were studied. The first set of reporter molecules consists of molecules that have two terminal pyridyl groups, the “D-set”, the smallest of which is 4,4′-dypyridyl (See FIG. 4). By isolating this set of molecules with the same terminal groups and similar structures, the relationship between the SERS enhancement and the molecule length can be more clearly observed. In particular, the 1600 cm⁻¹ C═C stretch and 1000 cm⁻¹ symmetric ring breathing modes were monitored. These particular modes were chosen because they are common to all of the molecules in the D-set, clearly distinguishable from the other molecular Raman modes, and strong enough to yield good signal to noise in both normal Raman and SERS spectra. For both Raman modes, the SERS enhancement factor of the molecules in the D-set decreased as the length of the molecule increased; a trend that is consistent with that observed in earlier experiments. There is much less scatter evident in this new set of data, which is probably due to similarities between all of the molecules in this set. The enhancement factor consistently decreases with increasing molecule size within the error of the experiment. This data strongly supports the contention that SERS enhancement can be significantly influenced by the ability of a reporter molecule to access the hotspots created by SERS-active materials.

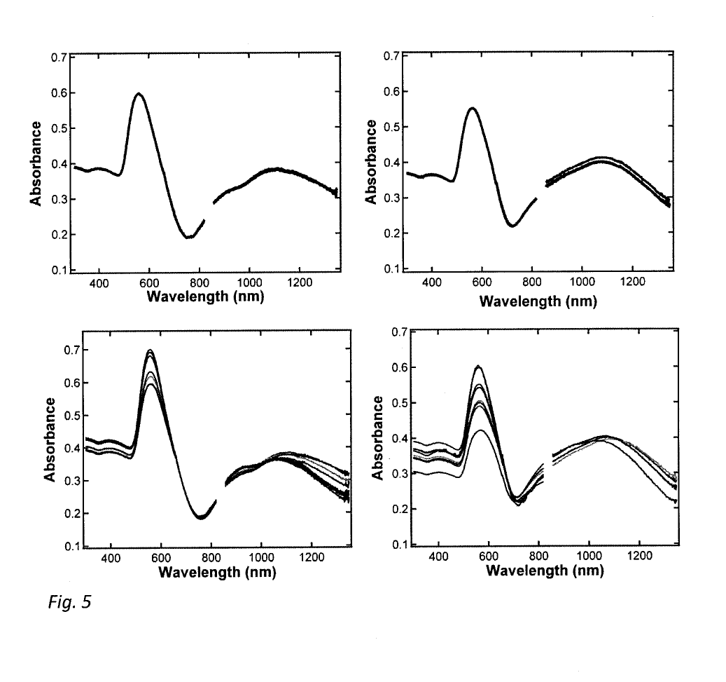

The same trend holds true for all of the excitation wavelengths studied—633, 785, and 1064 nm—further supporting the above conclusions. In order to compare data from different wavelengths, however, it is important to ensure that the data at each excitation wavelength comes from essentially the same sample. In other words, the sample should not continue to aggregate during the process of collecting the SERS and UV-vis-NIR measurements (See FIG. 5). The absorbance spectra taken at the beginning and the end of a series of measurements are essentially the same (FIG. 5, top), indicating that the state of aggregation remained relatively constant over the course of the measurements. Since the state of aggregation is the same for each SERS measurement, the geometry of the hotspots within the sample and, therefore the number of reporter molecules that can access the hotspots, should be the same as well. From sample to sample, there is greater variation evident in the absorbance spectra (FIG. 5, bottom), which probably causes some of the measured variation in SERS enhancement factors between samples. However, this variation is minimal, as evidenced by the small error bars in FIG. 4.

The consistent decrease in SERS enhancement factor with increasing reporter molecule length indicates that the observed enhancement depends on the ability of the molecule to fit into crevices formed by the aggregated colloid, and not the particular molecular or plasmon resonances that may be active in the system. First of all, the wavelength independence of the trend indicates that it is not influenced by any particular molecular resonance, since such a resonance would dramatically increase the SERS signal at only one of the excitation wavelengths used, not all three studied. Since no such anomalies are observed in the data, it may be concluded that molecular resonances do not impact the results. The observed trend also does not appear to depend on the plasmon resonance of the Au nanoparticles. The three excitation wavelengths used would excite different resonances of the particles. SERS data using the 633-nm laser excitation is primarily influenced by the higher energy plasmon resonance that is associated with either single particles or the transverse mode of particle aggregates (discussed in the Simulations section below). The SERS signals from the 785 and 1064-nm laser excitation, however, are influenced by the lower energy plasmon resonance that is associated with nanoparticle aggregates. Therefore, the decrease in SERS as a function of increasing molecule size does not depend on the plasmonic properties of the nanoparticle aggregates.

One aspect of the data that may be influenced by the plasmon resonance of the nanoparticle aggregates is the magnitude of the SERS enhancement factors observed. In this experiment, samples were optimized for SERS using 785-nm laser excitation, so it is unsurprising that the highest enhancements observed from these experiments result from using 785-nm excitation. The enhancements using 1064-nm excitation are fairly comparable to those from 785-nm excitation, which may be indicative of the fact that both of these enhancements would result from enhanced near-field associated with nanoparticle aggregates. In contrast, enhancements achieved using 633-nm excitations were significantly lower. As mentioned previously, the optical properties of these aggregates at 633 nm is mostly associated with the plasmon resonance of single particles, or from the transverse plasmon resonance mode of a nanoparticle aggregates. It is possible that these resonances do not create equally high near-field enhancements, or that they are not as efficient at scattering the Raman-shifted photon to the detector, an issue which will be addressed in a following section.

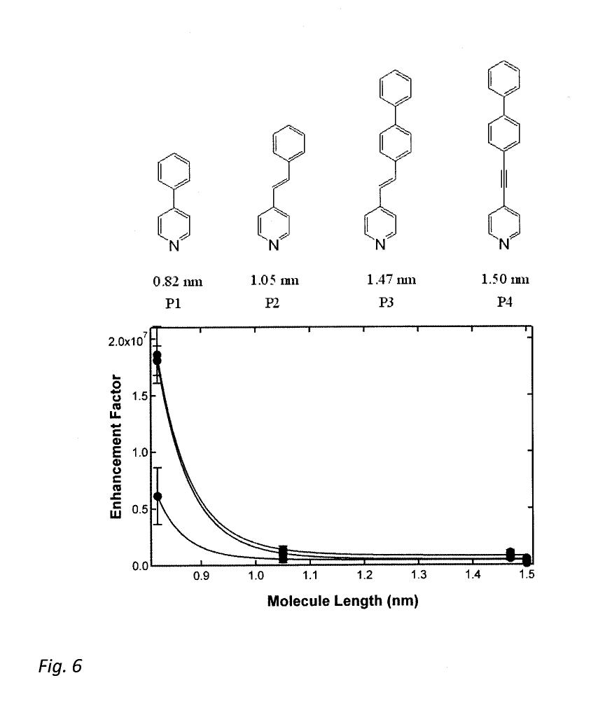

Another set of molecules was also studied to confirm that this effect was not unique to the D-set. This second set of molecules, the “P-set”, consists of molecules with one terminal pyridyl group and one terminal phenyl group, the smallest of which was 4-phenylpyridine (See FIG. 6). As with the D-set, the SERS enhancement factors observed from this set of molecules decreases with increasing molecule size, in a much more consistent manner than observed in the original experiments utilizing a varied mix of molecule compositions. Again, the observed enhancements from all three excitation wavelengths behaved in this manner, and the enhancements using 633-nm excitation were significantly lower than those using 785 and 1064-nm excitation. This set of molecules further supports the theory that SERS enhancements observed on aggregated colloid is strongly dependent on the ability of the molecule to fit into the crevices formed by the colloid.

One interesting and unexpected feature of this data set is that the enhancement observed from P3 is comparable to that observed from P2 at every excitation wavelength, despite the fact that the P3 is 40% longer. This apparent discrepancy illustrates one aspect of why there was significantly more scatter observed in the original experiments than in these experiments. In the P-set of molecules, the smallest two, P1 and P2, required the addition of salt to induce aggregation in the Au colloid, whereas the two larger molecules, P3 and P4, did not. This difference indicates that there are different mechanisms at work inducing aggregation in these samples. This difference, in turn, affects the types of aggregates and crevices formed, which may change the near-field enhancement produced in the hotspots and the ability of the molecules to access those hot spots. For the P-set, this effect seems to have resulted in a smaller decrease than expected between the molecules where the transition between aggregation mechanisms occurs. Since the molecules are still fairly similar, this effect did not seem to drastically affect the overall trend. In contrast, the varied molecules used in the original experiments probably interacted in very different ways with the Au colloid, resulting in the variability to the observed trend.

Enhancement vs. Colloid Size

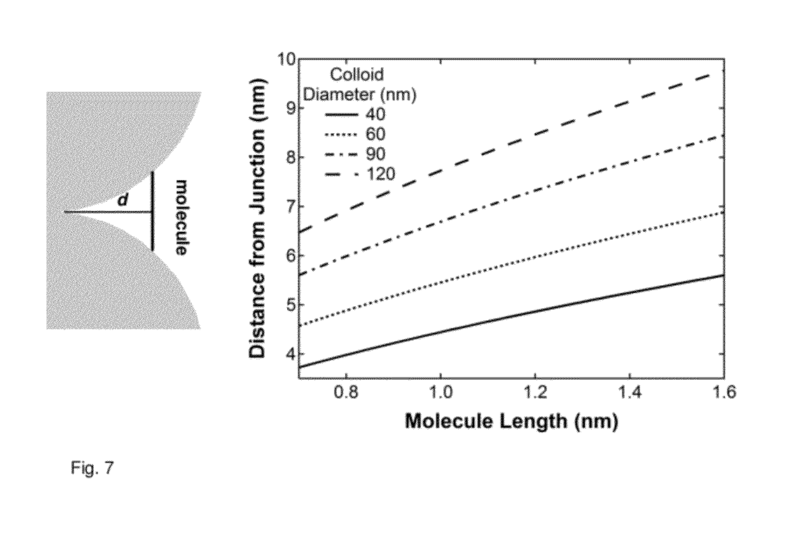

To probe this system further, the crevices formed by the aggregated Au colloid were changed in a controlled manner. One way to accomplish this is to use different sizes of colloid. The larger radius of curvature for larger colloid would result in deeper and sharper crevices formed between the particles. As illustrated in FIG. 7, the larger the colloid, the further away the molecules are situated. Also, the difference in distance from the junction between the smallest molecule (D1) and largest molecule (D6) in this study is greater for the larger colloid than the smaller colloid. So, to investigate the impact of the crevice geometry on the SERS enhancements achieved, four colloid sizes were investigated: 40, 60, 90, and 120 nm colloid. The maximal SERS enhancements achieved using a number of molecules (D1, D2, D3, D4, D5 and D6) in the D-set were obtained.

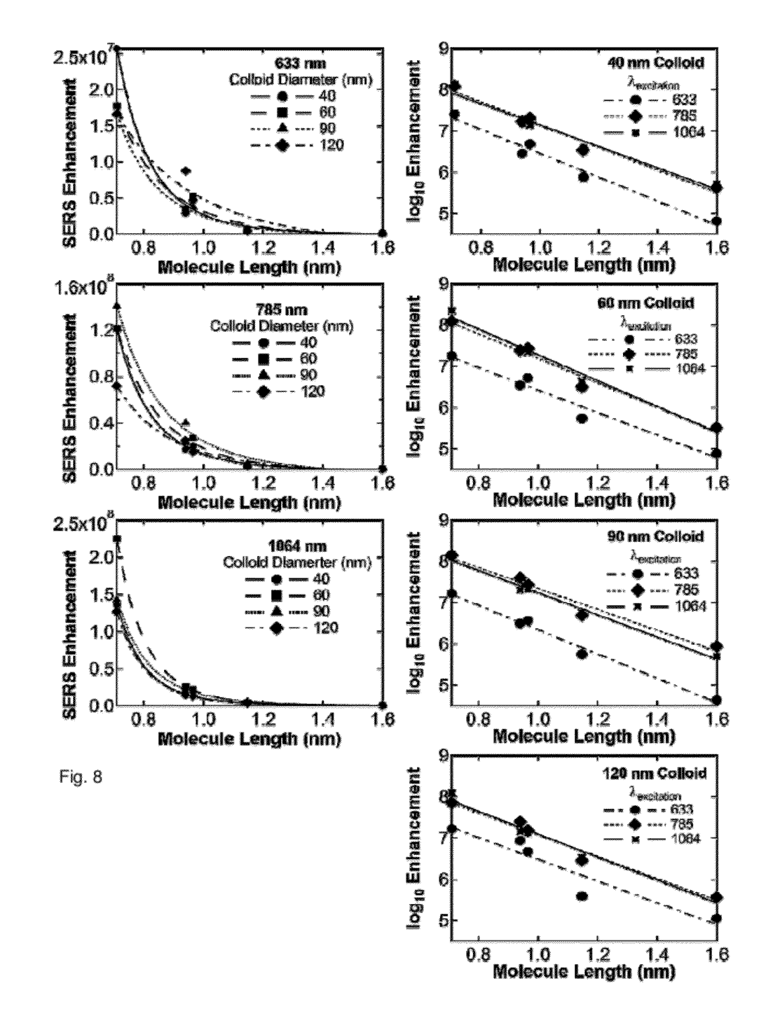

As FIG. 8 shows, very little difference was observed in the SERS enhancements arising from aggregates of the 4 different colloid sizes studied. On average, the different colloid sizes resulted in ˜30% variation in the SERS signals observed at each wavelength for each molecule. This variation was, for the most part, not systematic; no colloid size consistently yielded the greatest or the least SERS enhancements using 633 nm excitation. SERS enhancements using 785-nm excitation were always greatest using 90-nm colloid, although the SERS enhancements from the other colloid did not fall into any particular pattern. However, the enhancements observed using 1064-nm excitation did yield a consistent pattern. 60-nm colloid almost always yielded the brightest enhancements using 1064-nm excitation, followed by 90-nm, 40-nm, and then 120-nm colloid. It may be that the data using 1064-nm excitation is more systematic because the excitation and Raman scattered wavelengths are only influenced by the plasmon resonance of the nanoparticle aggregates. In contrast, the single particle plasmon resonance will influence 633 and (to a lesser extent) 785-nm excitation, which may contribute to the observed SERS signals in a variety of ways.

When different samples were fabricated to maximize the SERS enhancements observed at each of the three wavelengths studied, the greatest enhancements observed on each colloid size consistently came from D1 using 1064-nm excitation. For the other molecules, the enhancements observed using 785 and 1064-nm were comparable, as was observed in the previous experiments. Also, 633-nm excitation again yielded significantly weaker enhancements than the other two wavelengths.

One interesting feature of the data collected is that for each colloid size, the rate at which the SERS enhancement decreases as a function of colloid size is approximately equal for each excitation wavelength used. The slopes of the linear fits shown in FIG. 8 (right) differ by only ˜6%. This fact may again support the theory that the data is mainly a result of geometric considerations, and not plasmonic ones.

### Finite Element Simulations of Electromagnetic Properties

Electromagetic simulations were performed using COMSOL Multiphysics 3.4 to develop a deeper understanding of electromagnetic near-field enhancements created by the crevices in aggregated Au nanoparticles of different sizes. COMSOL uses the finite element method to calculate the optical properties of a nanoparticle. This tool splits user-defined, finite simulation geometry into many discrete elements, and solves Maxwell's equations in the simulation volume using the dielectric function of the materials within and the boundary conditions applicable to the interfaces between the constituent materials. It is particularly useful for calculating the optical properties of particles of arbitrary shape for which analytical solutions (like Mie Scattering Theory) do not exist.

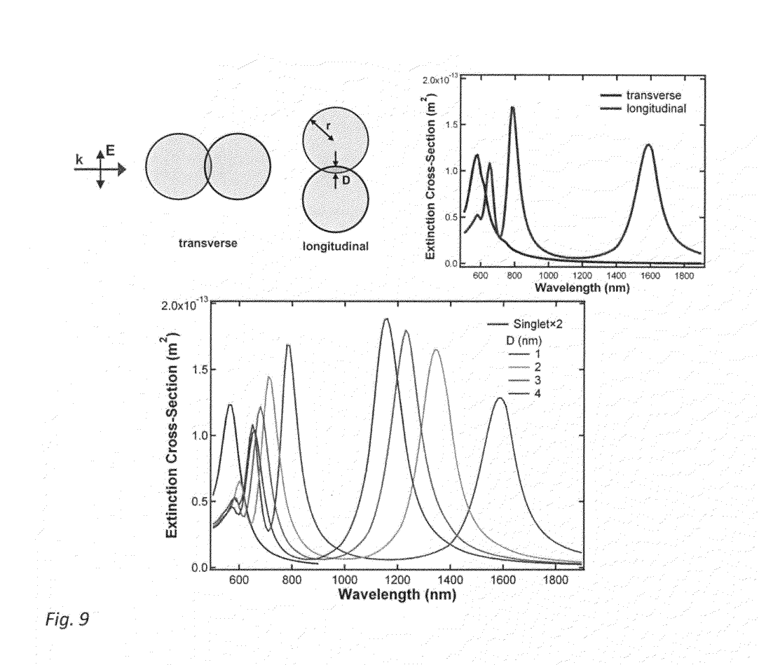

In the described experiments, calculations were focused on the optical properties of Au dimers. It has been shown that nanoparticle dimers generally exhibit near-field enhancements orders of magnitude greater than single particles, so it is reasonable to assume that most of the SERS intensity observed arises from nanoparticle aggregates. Although previous experience has shown that samples created by aggregating Au colloid contain a wide distribution in the types and sizes of aggregates formed, Au nanoparticle dimers were studied to reduce computational complexity. Previous electron microscopy data also suggests that the aggregates in the samples consist of nanoparticles which are touching. Since experimentally fabricated colloid is not perfectly spherical, the nanoparticles are in contact over some finite area (i.e. not at a single point). As such, the contact area of each dimer is modeled by a small overlap in the constituent nanospheres- the smaller the overlap, the smaller the contact area. The simulations of FIG. 9 demonstrates that the broad plasmon peak in the near-IR that was evident in UV-vis measurements (see FIG. 5) can be attributed to aggregates in the solution, whereas the plasmon resonance centered about 600 nm contains contributions from both single particles and aggregates. The broadness in the aggregate peak could be a result of the variation in the contact area in each aggregate and/or the range in the number of nanoparticles present in each aggregate. The simulations show that the longitudinal plasmon, which is excited when incident light is polarized along the axis of the dimer particle, is very sensitive to the overlap of the two nanoparticles in the dimer, a feature which has been demonstrated previously with core-shell nanoparticles.

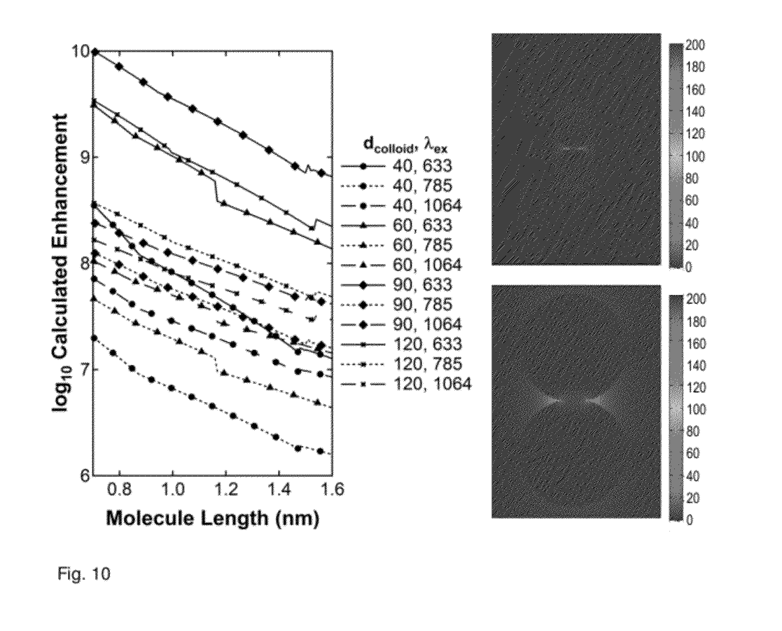

Since the calculated extinction spectra of overlapping dimers are comparable to experimental results, the same simulations were used to understand the near-field enhancements experienced by molecules situated in the crevices. It was assumed that the molecules that are experiencing the greatest near-field enhancement are situated parallel to the dimer axis, and are bound to each nanoparticle in the dimer at either end of the molecules, as depicted in FIG. 7. Making this assumption, the expected electromagnetic Raman enhancement of the 1600 cm⁻¹ peak at the center of the molecule may be calculated as a function of molecule length for the 4 different sizes of colloid considered (FIG. 10). To compare the results from the different sizes of colloid, the relative geometry was kept constant by setting the overlap in each case to be D=r_{colloid}/15. This overlap was chosen to approximate the location of the aggregate peak in the experimental UV-vis spectra well. The electromagnetic Raman enhancement was calculated as E²ₗₐₛₑᵣ×E²_{Raman} where Eₗₐₛₑᵣ and E_{Raman} are the near-field enhancements at the laser and Raman shifted wavelengths, respectively. Small kinks in the calculated trends occur at the boundaries between mesh elements in the simulation.

Qualitatively, simulations agree with the experimental data: the enhancement drops off as a function of molecule length and the rate at which it decreases is comparable for all colloid sizes and excitation wavelengths. The simulations also show that the rate of the enhancement decrease is roughly the same for all of the colloid sizes, excitation wavelengths, and Raman peaks examined. Although geometric factors contribute to a molecule's ability to fit into the crevice created by aggregated colloid of different sizes, the SERS enhancement also depends on the nature of the near-field enhancement created at the crevice.

Dimers that consist of larger colloids create near-fields that extend further away from the dimer junction at the wavelengths of interest. Thus, although a large molecule may have difficulty getting very far within the crevice created by a 120 nm Au colloid dimer, the near-field it experiences is still significantly enhanced since that near-field extends farther away from the dimer junction.

One interesting aspect of the described simulations that is not consistent with experimental results is that the SERS enhancement using 633-nm excitation is calculated to be much greater than that observed with 785 and 1064-nm excitation. In experiments, the enhancement using 633-nm excitation is significantly lower. One possible explanation for these results is that the optical properties of the sample are blocking the SERS scattered light, either through absorption or multiple scattering events that tend to scatter the light away from the detector in the measurement geometry used. More detailed studies of the absorption and angle-dependent scattering of aggregates would be required to develop further understanding of this issue.

### Example 1

### Conclusion

The impact of reporter molecule size on SERS enhancements has been studied using aggregated Au colloid. By studying sets of reporter molecules with similar structure and identical terminal functional groups, the experiments with each molecule can be more readily compared, since parameters such as the mechanism of aggregation, reporter orientation, and reporter affinity will be fairly consistent within the set. Experiments with two sets of reporter molecules, three excitation wavelengths, and four Au colloid sizes all demonstrated that the SERS enhancement factor achieved decreased with reporter molecule size. The consistency of the trends with variation in each of these parameters supports the conclusion that the effect is based on the molecule size, not particular plasmon or molecular resonances. Electromagnetic simulations of Au dimers indicate that these results are consistent with the decrease in the electromagnetic near-field created in the crevices between the Au colloids as the distance from the particle junction increases. These results demonstrate that molecule size is a critical parameter to be considered when designing a system to generate the highest possible SERS enhancements.

### Example 2

Introduction

As described above, one way of increasing the strength of a SERS signal from any SERS system is to use reporter molecules with very high SERS activity. Many potential reporter molecules have been screened, and certain conclusions about the structure/activity relationship have been made, including but not limited to:

a) A reporter molecule with high SERS activity may have at least one aromatic ring conjugated with one or more unsaturated groups. This extended conjugated system increases the Raman cross-section of the molecule.

b) A reporter molecule with high SERS activity may have a functional group capable of forming a stable bond with the gold surface, in a way that it positions the molecule in an orientation that is perpendicular to the surface. The best and more consistent binding groups are pyridyl, monosubstituted ethynyl, and in a lesser extent, thiol groups. Thiol groups, although capable of forming strong bonds with gold, appear to react very slowly.

c) In general, the presence of a second binding functional group in the opposite end of the molecule results in larger enhancement.

### Solution Raman Spectra

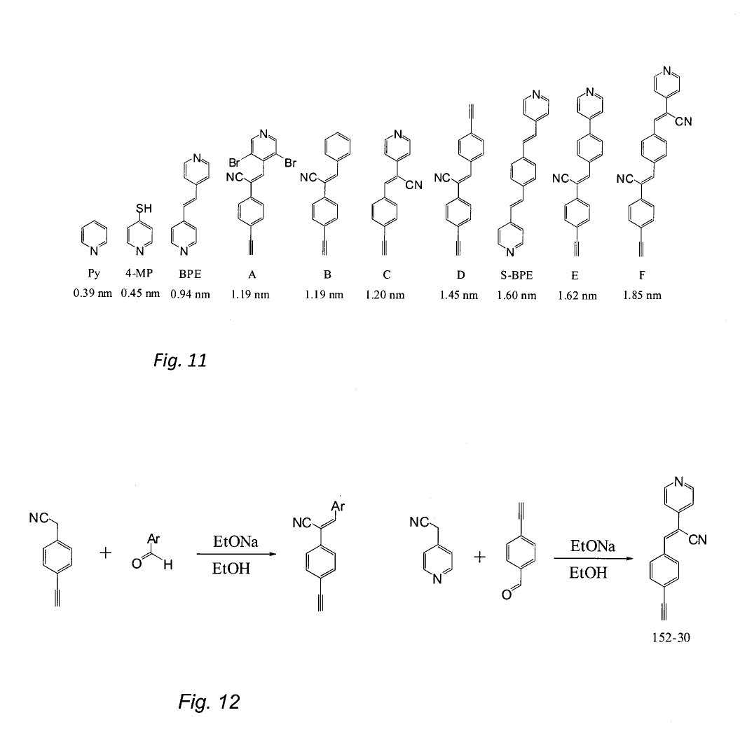

Experiments were performed as described herein to determine if the Raman cross-sections of a set of molecules correlate with the corresponding extension of the conjugated system of those molecules. FIG. 11 shows the set of molecules chosen for this aspect of the study. The chosen reporter molecules range in size from a single aromatic ring (pyridine) to a molecule that contains three aromatic rings bridged by ethylenic groups and having also an alkynyl and 2 cyano groups, all groups conjugated (molecule F). The molecules were chosen to have pyridyl and/or ethynyl groups to bind to the gold surface, while belonging to the same structural family of compounds. A reporter was included in which one of the rings is forced out of the plane by the presence of two Br substituents in a pyridyl ring (molecule A) to explore the effect of decreased conjugation, and another molecule was included without a metal binding functional group in one of its ends. Pyridine, 4-mercaptopyridine (4-MP) and BPE were purchased from Sigma-Aldrich. Bis-(2-pyrid-4-ylethenyl)benzene (S-BPE) was purchased from Exciton. The rest of the molecules were prepared in house, by Knovenagel condensation between 4-ethynylphenyl acetonitrile and the corresponding aldehyde. Molecule C was synthesized by the condensation of 4-pyridylacetonitrile and 4-ethynylphenylbenzaldehyde (See FIG. 12).

FIG. 11 also shows the corresponding approximate size of each of the molecules, calculated with ArgusLab, a software that utilizes semiempirical methods for geometry optimizations.

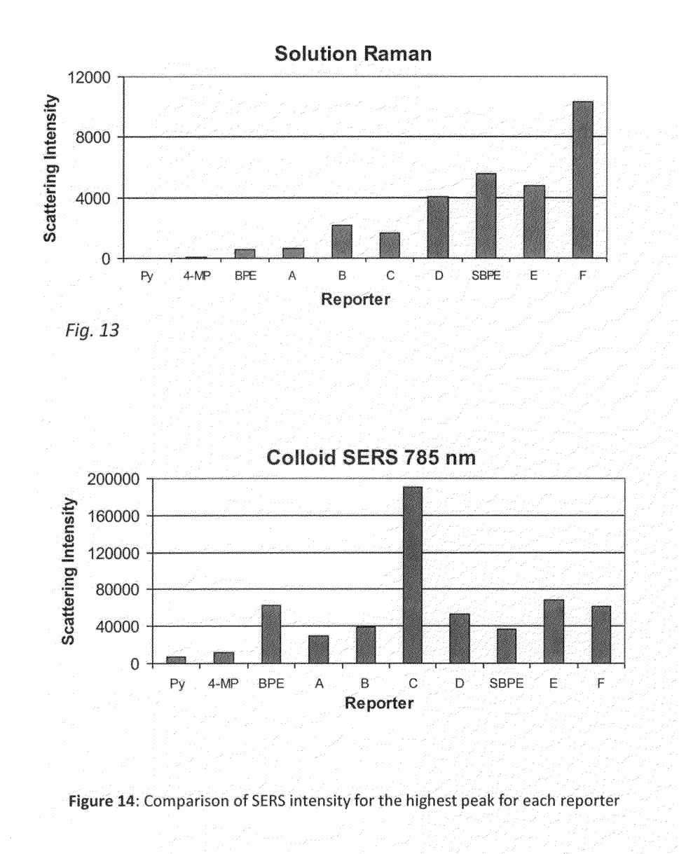

The Raman spectra of this experiment were obtained from 0.1 M solutions of the reporter in DMF, using pure DMF as the reference (all the Raman and SERS readings were carried out using an Ocean Optics QE6500 785 nm spectrometer). The exceptions were pyridine and 4-mercaptopyridine, which due to sensitivity limitations were obtained with a 1 M solution, and S-BPE and molecule F, which because of limited solubility were performed with a 0.02 and 0.01 M solution respectively. All readings were later corrected for concentration. The Raman intensity results are plotted in FIG. 13.

It can be seen from the graph of FIG. 13 that the relative Raman intensities follow the expected trend. The Raman signal increases with the size of the conjugated path in the molecule. The main exception is molecule A. The lower Raman intensity of this molecule compared to others of similar size is likely due to the effect that the presence of the two Br groups has on the planarity of the molecule. The intensities of the Raman spectra of the four largest molecules are very high, and large increases take place with small changes in the size of the molecules. This clearly indicates that the Raman cross-sections of the reporters increase with the extension and efficiency of the conjugated system.

### Colloid SERS Experiments

Colloid SERS experiments were carried out as follows: 7 ul of 0.1 mM solution of the reporter molecule (except pyridine, vide infra) is added to a vial containing 1 mL of 90 nm /gold colloid (2× concentration) and the vial is immediately shaken (It having been determined as described above that this amount of reporter gives the maximum SERS possible, except for pyridine. In this case, because of the high solubility of pyridine in water, it is likely that only a small fraction of it binds to the gold surface, resulting in the need to add more reporter: 20 ul of a 1 mM solution. Since this amount results in the highest SERS possible, it is estimated that the amount of pyridine molecules attached to the gold is comparable to the other reporters, which is relevant when calculating the enhancement factors). If aggregation takes place, SERS is read continuously with 1 sec integration until the highest SERS is obtained. The spectrum is then saved. If no aggregation takes place, 20 ul of NaCl 1 M is added to induce aggregation followed by SERS reading. Of this group of compounds, pyridine and molecules A, B and D needed the addition of NaCl to induce aggregation. Shorter integration times were needed for compounds C, E and F because the signals for these reporter molecules went out of scale when irradiated for 1 sec. The SERS intensities of the molecules requiring shorter integration times were corrected afterwards accordingly. The graph in FIG. 14 shows the height of the tallest peak of the SERS spectrum for each of the reporters.

It can be observed in the FIG. 14 graph that SERS intensity increases from pyridine to BPE, and dramatically to molecule C. The SERS intensity is lower for molecule A, which is in line with the result obtained for the solution Raman spectrum. The low SERS of molecule B is of no surprise since it has been previously observed that the lack of a metal binding group in one of the molecular ends usually results in lower SERS activity than expected. Contrary to the solution Raman experiments, the SERS intensities for the largest molecules are much lower than molecule C, and the increase of SERS due to molecular size seems to come to a halt. It is clear that the surface Raman enhancement for larger molecules does not correlate with higher cross-section. To better understand this behavior, SERS experiments using a commercial SERS substrate, Klarite™ were performed.

### Substrate SERS Experiments

Experiments on Klarite substrates were performed to determine the reasons for the low colloid SERS response obtained with the largest reporters. Klarite substrates are nano-engineered gold chips that provide the necessary roughened surface for SERS enhancement.

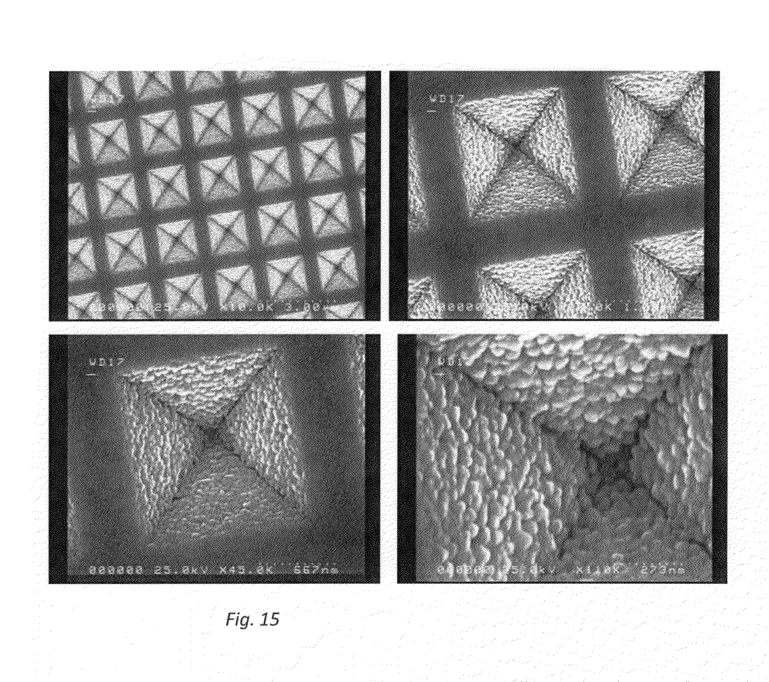

SEM images of the substrates were obtained as shown in FIG. 15. The surface appears to consist of pyramidal features of around 1.5 microns carved into the gold surface. The sides of these features are covered with irregular granules 30-40 nm in size. The rough surface formed by these granules is likely responsible for the SERS enhancement activity. It is important to note that the main difference between these substrates and the gold colloids discussed above is that with the substrates the addition of reporter molecules should not induce any change in the structure of the surfaces, while the addition of reporter to a colloid usually induces aggregation. This induced contact between particles is the responsible for the high SERS enhancements.

The experiments were run as follows: An adhesive rubber gasket containing a hole of around 2 mm across and 3 mm deep is adhered to the gold substrate. The resulting cavity can hold ˜10 ul of a liquid. 10 ul of a 10 mM solution of the reporter in 1/9 DMF/EtOH is added to the cavity and left there for 1 minute. Then the solution is pipetted off, and the surface is rinsed with 10 ul of EtOH. The ethanol is discarded and the SERS is read on the 785 nm spectrometer. The spectrum is recorded, and the substrate is treated again with 10 mM reporter solution and rinsed as mentioned above. SERS is read again. The process is repeated until no more increase in SERS takes place. It typically takes 3 to 5 applications to get to constant SERS. Pyridine did not show any SERS, while molecule F had to be performed with a 1 mM solution due to solubility limitations. The described method was designed to assure saturation of the surface with the reporter molecule. To assure that no signal is due to the Raman spectrum of solid formed on the surface (non-SERS) a few control experiments were done on the smooth part of the gold chip, resulting in negligible Raman signal.

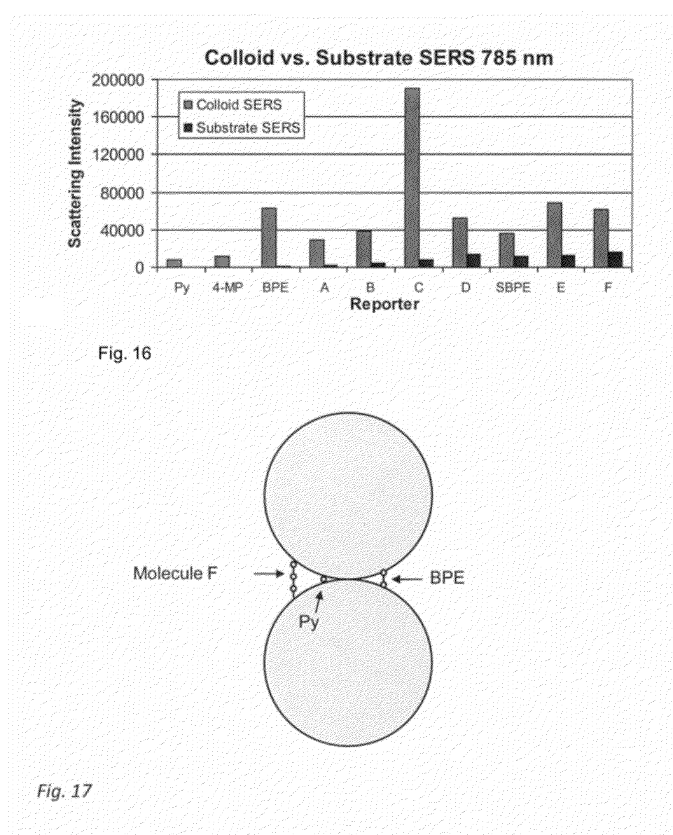

The scattering intensity results of the substrate SERS experiments are shown in FIG. 16.

Two clear observations may be made from the results illustrated in FIG. 16. (a) The use of gold colloid results in much higher surface Raman enhancement than the substrate; and (b) the SERS signal in the substrate increases with the size of the molecule. In the case of molecule A the SERS signal is not as strong as observed for similar sized molecules due to the decrease of molecular planarity, and except for the difference between molecules B and C (which have comparable molecular size), the relative SERS intensities of the whole series fall in the expected order. Here again the higher intensity for molecule B than BPE is observed. Reporter F was the highest even when though a lower concentration solution for the experiment was used. It is also evident that the increase of the signal is not proportional to the increase of the solution Raman intensities. It is believed that the molecules stand perpendicular to the surface, and that the increase of the distance from the farthest end of the molecule to the gold surface results in a lower exposure of that side of the molecule to the enhanced electromagnetic field. Based upon the foregoing observation, it may be estimated that even longer conjugated molecules would not result in important increases in SERS activity and that at some point the SERS intensity should reach a plateau.

The results obtained from the gold substrate experiments suggest that the larger reporters have the potential to give higher SERS response. Thus, there must be other factors affecting the SERS activity of these same molecules when used with gold colloids. As described above, most of the colloid SERS signal is generated in hot spots present in the join area between two particles that have aggregated. It is possible that the reporter molecule positions itself between the particles, acting as a bridge between them. For larger molecules this means that the two particles must stay further from each other. The larger separation of the particles results in lower electromagnetic enhancement, hence producing lower SERS signal.

A more likely theory is that when two particles aggregate the particles basically become fused, and the reporter molecule positions itself in the tiny crevice formed next to the particle-to-particle contact area. Theoretical calculations show that the electromagnetic field is very strong in this area, but it quickly decreases as the distance to the contact area becomes longer (and the gap between the particles becomes larger). This suggests, as described in detail above and schematically illustrated in FIG. 17, that smaller molecules will be able to penetrate deeper in this crevice and show larger enhancement, while larger molecules will be forced to stay further outside, where the molecule can maintain a vertical orientation while bridging the two particles. As a result, the high Raman cross-section of a longer molecule will be offset by the lower EM field to which it is exposed due to its placement further out in the crevice. If this is the case, it would be expected that a higher enhancement factor would be observed for smaller molecules like pyridine, and a poorer factor for large molecules such as molecule F.

It is important to remember that the Klarite substrates also have nano-sized granules that may be in contact with each other. Substrate fabrication methods (probably vapor deposition) may result however in different geometries without the type of crevices that are found in aggregated colloid. Thus, the substrate would be expected to result in similar electromagnetic enhancement for every reporter regardless of its size.

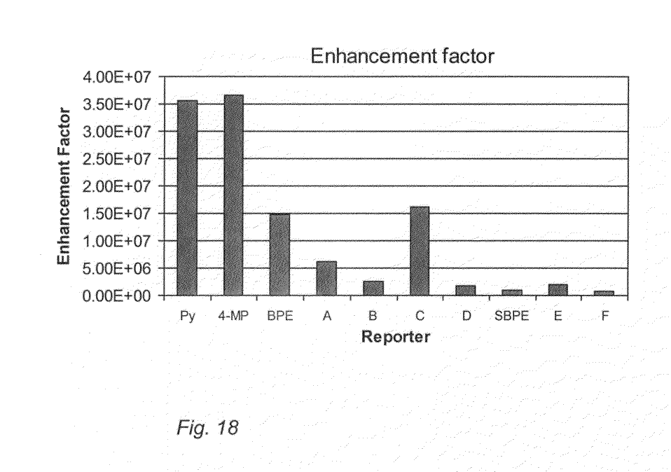

The enhancement factors for the colloid SERS experiments of all the molecules in the series were calculated, and the results are shown in FIG. 18. The enhancement factor was calculated by using the following formula:

E=[S(Cᵣ/Cₛ)]/R

in which E is the enhancement factor, S the colloid SERS intensity, R the solution Raman intensity, and Cᵣ and Cₛ are the concentrations of the reporter in the Raman and SERS samples respectively. The enhancement factor is the highest for the smallest molecules, pyridine and 4-mercaptopyridine, and except for C, the enhancement factor quickly goes down for larger molecules. These results are in line with the theory described above. The decrease of enhancement with the size of the molecule presents a challenge to the search for bright reporter molecules and suggests that the most efficient way to prepare highly bright tags is by using resonant reporters. This last option is a valid one, but resonant reporters are usually large, more complex molecules that result in too many bands and high backgrounds, something that may not be convenient when a few discrete peaks are what it is desired.

### Example 2

### Conclusions

The side by side comparison of data from Raman solution, colloid SERS and substrate SERS reveals that the lower than expected SERS brightness of the larger conjugated molecules may not be related to intrinsic properties of the molecules. Instead, the geometry of the aggregated particles puts constrains to these molecules to access areas of higher EM energy. This clearly sets limitations to the search for brighter reporters because higher Raman cross-sections of longer conjugated systems will be offset by the lower enhancement.

The lower SERS of molecule A is explained by the deviation of planarity of the pyridine ring. On the other hand, the low colloid SERS and higher substrate SERS of B may be due to the lack of bridging capability between particles, resulting in an unfavorable position and/or orientation of the reporter for efficient enhancement.

### General Conclusions

It may be concluded from the molecular-size/SERS-enhancement studies described above that the shorter the reporter molecule, the higher the SERS enhancement is when the molecule is added to gold colloid. On the contrary, as demonstrated in the Solution Raman spectra experiments, longer molecules, i.e. molecules having a larger conjugated path have a larger Raman cross section and therefore exhibit greater Raman signal intensity in solution. When these two separate effects are combined in a colloid system an offset is seen to occur. In particular, the enhancement of SERS signal of generally greater for smaller reporter molecules as described above, but this is partially offset because small molecules have low Raman cross-section. Thus, SERS intensity as a function of reporter molecule length actually increases with the size of the molecule up to a size of ˜1.2 nm, when the reporter is adsorbed to the surface of nano-scale enhancing colloids. Reporter molecules that are longer have a higher Raman cross-section, but their SERS enhancement decreases considerably, resulting in a lower SERS intensity. Accordingly, reporter molecules with 2 or more metal binding groups that are no more than 1.2 nm apart, but with higher conjugated systems would have much higher SERS brightness. This increased conjugation would arise from an extended conjugated path towards the sides of the molecule. Some examples are illustrated below. The larger molecular surface of this type of reporters may result in an increased tendency to adsorb flat to the metal surface. For this reason, the presence of more binding groups would be favorable for both, a stand up binding position and better binding stability to the metal surface.

A list of molecules with these characteristics and which should exhibit exceptional SERS brightness includes, but is not limited to, 1,8-diethynyl-4,5-bis(4-pyridylethenyl)anthracene, 1,8,9-triethynyl-10-(4-pyridylethenyl)anthracene, 6,13-bisethynylpentacene, 5,7,12,14-tetraethynylpentacene, 9,10-bisethynylanthracene, 1,4,5,8,9,10-hexaethynylanthracene, 1,8-bisethynylanthracene, 9-ethynyl-10-(4-pyridylethynyl)anthracene, 1,8,9-tris(4-pyridyl)anthracene, 1,8-bis(4-pyridyl)-10-(4-pyridylethynyl)anthracene, 1,8-bis(4-pyridyl)-10-ethynylanthracene, 1,8-bis-(4-pyridyl)-10-(4-pyridylazo)anthracene, and 1,8-diethynyl-10-(4-pyridylazo)anthracene.

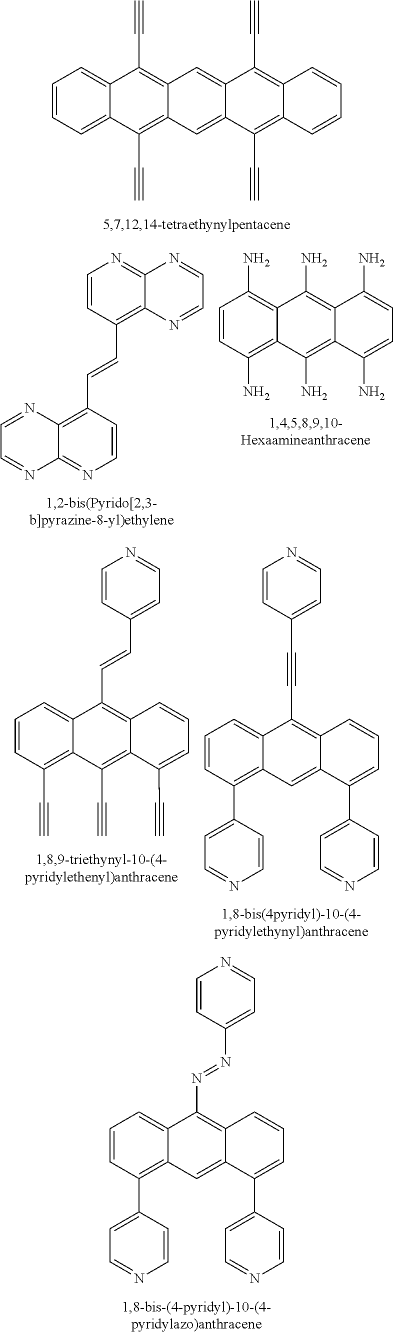

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

While the embodiments disclosed herein have been particularly shown and described with reference to a number of examples, it would be understood by those skilled in the art that changes in the form and details may be made to the various embodiments disclosed herein without departing from the spirit and scope of the disclosure and that the various embodiments disclosed herein are not intended to act as limitations on the scope of the claims.

All references sited herein are incorporated in their entirety by reference for all matters disclosed therein.

What is claimed is:

1. A SERS reporter molecule comprising:
a reporter molecule having a length sufficiently short to fit into a crevice formed by the enhancing surfaces of adjacent enhancement particles; and
a conjugated path length which is as large as possible, provided the overall molecule length is maintained sufficiently short to fit into the crevice formed by the enhancing surfaces of adjacent enhancement particles.

2. The SERS reporter molecule of claim 1 wherein the reporter molecule length is selected or fabricated to be less than or equal to 1.2 nm.

3. The SERS reporter molecule of claim 1 wherein the reporter molecule comprises one of 1,8-diethynyl-4,5-bis(4-pyridylethenyl)anthracene, 1,8,9-triethynyl-10-(4-pyridylethenyl)anthracene, 6,13-bisethynylpentacene, 5,7,12,14-tetraethynylpentacene, 9,10-bisethynylanthracene, 1,4,5,8,9,10-hexaethynylanthracene, 1,8-bisethynylanthracene, 9-ethynyl-10-(4-pyridylethynyl)anthracene, 1,8,9-tris(4-pyridyl)anthracene, 1,8-bis(4-pyridyl)-10-(4-pyridylethynyl)anthracene, 1,8-bis(4-pyridyl)-10-ethynylanthracene, 1,8-bis-(4-pyridyl)-10-(4-pyridylazo)anthracene, and 1,8-diethynyl-10-(4-pyridylazo)anthracene.

4. The SERS reporter molecule of claim 1 further comprising at least one aromatic ring conjugated with one or more unsaturated groups.

5. The SERS reporter molecule of claim 1 further comprising two or more metal binding groups.

6. The SERS reporter molecule of claim 5 further comprising two or more metal binding groups that are no more than 1.2 nm apart.

7. The SERS reporter molecule of claim 1 comprising at least one functional group capable of forming a stable bond with the enhancing surface, such that the bond positions the molecule in an orientation that is perpendicular to the enhancing surface.

8. The SERS reporter molecule of claim 7 wherein the functional group comprises one of pyridyl, monosubstituted ethynyl or a thiol group.

9. A SERS tag comprising:
a core comprising at least two aggregated particles of a SERS enhancing material wherein the contact point between the particles defines a crevice; and
a reporter molecule having a length sufficiently short to fit into the crevice and a conjugated path length which is as large as possible, provided the overall reporter molecule length is maintained sufficiently short to fit into the crevice.

10. The SERS tag of claim 9 wherein the reporter molecule length is selected or fabricated to be less than or equal to 1.2 nm.

11. The SERS tag of claim 9 wherein the reporter molecule comprises one of 1,8-diethynyl-4,5-bis(4-pyridylethenyl)anthracene, 1,8,9-triethynyl-10-(4-pyridylethenyl)anthracene, 6,13-bisethynylpentacene, 5,7,12,14-tetraethynylpentacene, 9,10-bisethynylanthracene, 1,4,5,8,9,10-hexaethynylanthracene, 1,8-bisethynylanthracene, 9-ethynyl-10-(4-pyridylethynyl)anthracene, 1,8,9-tris(4-pyridyl)anthracene, 1,8-bis(4-pyridyl)-10-(4-pyridylethynyl)anthracene, 1,8-bis(4-pyridyl)-10-ethynylanthracene, 1,8-bis-(4-pyridyl)-10-(4-pyridylazo)anthracene, and 1,8-diethynyl-10-(4-pyridylazo)anthracene.

12. The SERS tag of claim 9 further comprising at least one aromatic ring conjugated with one or more unsaturated groups.

13. The SERS tag of claim 9 further comprising two or more metal binding groups.

14. The SERS tag of claim 13 further comprising two or more metal binding groups that are no more than 1.2 nm apart.

15. The SERS tag of claim 9 comprising at least one functional group capable of forming a stable bond with the enhancing surface, such that the bond positions the molecule in an orientation that is perpendicular to the enhancing surface.

16. The SERS tag of claim 15 wherein the functional group comprises one of pyridyl, monosubstituted ethynyl or a thiol group.

17. A method of selecting a SERS reporter molecule to optimize the enhancement of the SERS signal provided by said reporter molecule upon excitation in association with a particulate or colloidal enhancing surface, said method comprising:
selecting or fabricating a reporter molecule length to be sufficiently short to fit into a crevice formed by the enhancing surfaces of adjacent enhancement particles; and
selecting or fabricating the reporter molecule to have as high a conjugated path length as is possible provided the overall molecule length is maintained sufficiently short to fit into the crevice formed by the enhancing surfaces of adjacent enhancement particles.

18. The method of claim 17 wherein the reporter molecule length is selected or fabricated to be less than or equal to 1.2 nm.

19. The method of claim 17 wherein the reporter molecule is one of 1,8-diethynyl-4,5-bis(4-pyridylethenyl)anthracene, 1,8,9-triethynyl-10-(4-pyridylethenyl)anthracene, 6,13-bisethynylpentacene, 5,7,12,14-tetraethynylpentacene, 9,10-bisethynylanthracene, 1,4,5,8,9,10-hexaethynylanthracene, 1,8-bisethynylanthracene, 9-ethynyl-10-(4-pyridylethynyl)anthracene, 1,8,9-tris(4-pyridyl)anthracene, 1,8-bis(4-pyridyl)-10-(4-pyridylethynyl)anthracene, 1,8-bis(4-pyridyl)-10-ethynylanthracene, 1,8-bis-(4-pyridyl)-10-(4-pyridylazo)anthracene, and 1,8-diethynyl-10-(4-pyridylazo)anthracene.

20. The method of claim 17 wherein the reporter molecule comprises at least one aromatic ring conjugated with one or more unsaturated groups.

21. The method of claim 17 further comprising selecting or fabricating the reporter molecule to include two or more metal binding groups.

22. The method of claim 21 further comprising selecting or fabricating the reporter molecule to have two or more metal binding groups that are no more than 1.2 nm apart.

23. The method of claim 17 wherein the reporter molecule comprises at least one functional group capable of forming a stable bond with the enhancing surface, such that the bond positions the molecule in an orientation that is perpendicular to the enhancing surface.

24. The method of claim 23 wherein the reporter molecule comprises one of pyridyl, monosubstituted ethynyl or a thiol group.
